SEQUENCE LISTING (24 sequences)

SEQ ID NO: 1 - DNA, 23 bp, synthetic construct
  source 1..23: mol_type = other DNA
cgatgcctatggaaaccgcttgg

SEQ ID NO: 2 - DNA, 22 bp, synthetic construct
  source 1..22: mol_type = other DNA
ctcttctgaacagctacctgag

SEQ ID NO: 3 - DNA, 23 bp, synthetic construct
  source 1..23: mol_type = other DNA
tcaacctaccagagacctacctg

SEQ ID NO: 4 - DNA, 21 bp, synthetic construct
  source 1..21: mol_type = other DNA
tcctggaacatactccggtag

SEQ ID NO: 5 - protein, 15 aa, synthetic construct
LTVERLVSIGYPQEL

SEQ ID NO: 6 - protein, 15 aa, synthetic construct
GFNFIRGSQVAVQKR

SEQ ID NO: 7 - protein, 15 aa, synthetic construct
TNCPRYTSCETGFKD

SEQ ID NO: 8 - protein, 15 aa, synthetic construct
KDGDLFMSYRSMFQD

SEQ ID NO: 9 - protein, 15 aa, synthetic construct
DWVHYKGSLKEKTVE

SEQ ID NO: 10 - protein, 15 aa, synthetic construct
PLATRNRTSVDFKDT

SEQ ID NO: 11 - protein, 15 aa, synthetic construct
LATRNRTSVDFKDTD

SEQ ID NO: 12 - protein, 15 aa, synthetic construct
HQLTRSISEIKPPNL

SEQ ID NO: 13 - protein, 15 aa, synthetic construct
LTVERLVSIGYPQEL

SEQ ID NO: 14 - protein, 15 aa, synthetic construct
KFIQRDDTERFYILN

SEQ ID NO: 15 - protein, 15 aa, synthetic construct
GSLFRSGSRQTLFAS

SEQ ID NO: 16 - protein, 15 aa, synthetic construct
PLATRNRTSVDFKDT

SEQ ID NO: 17 - protein, 15 aa, synthetic construct
HQLTRSISEIKPPNL

SEQ ID NO: 18 - protein, 15 aa, synthetic construct
GIVYSGGSSDTICDL

SEQ ID NO: 19 - protein, 15 aa, synthetic construct
MMGSLFRSGSRQTLF

SEQ ID NO: 20 - protein, 15 aa, unidentified
YTLAVYKSPEYESLG

SEQ ID NO: 21 - protein, 15 aa, synthetic construct
VDINEMESPLATRNR

SEQ ID NO: 22 - protein, 15 aa, synthetic construct
VGKVFLATNSDYKTY

SEQ ID NO: 23 - protein, 15 aa, synthetic construct
PLFFGEGTVLRQVDT

SEQ ID NO: 24 - protein, 15 aa, synthetic construct
TGKLKIGTYTGPLQH

(19) United States
(12) Patent Application Publication
(10) Pub. No.: US 2025/0320562 A1
(43) Pub. Date: Oct. 16, 2025

(54) ALTERNATIVELY SPLICED ISOFORM IN CANCER AND METHODS OF USE THEREOF

(71) Applicant: THE CHILDREN'S HOSPITAL OF PHILADELPHIA, Philadelphia, PA (US)

(72) Inventors: Andrei THOMAS-TIKHONENKO, Philadelphia, PA (US); Manuel TORES-DIZ, Philadelphia, PA (US)

(73) Assignee: THE CHILDREN'S HOSPITAL OF PHILADELPHIA, Philadelphia, PA (US)

(21) Appl. No.: 18/866,685

(22) PCT Filed: Jun. 12, 2023
(86) PCT No.: PCT/US2023/025031

§ 371 Date: Nov. 18, 2024

Related U.S. Application Data
(60) Provisional application No. 63/351,244, filed on Jun. 10, 2022.

(51) Int. Cl. C12Q 1/6886; A61K 31/4745; A61K 31/52; A61K 31/5377; A61K 31/7056; A61K 31/7076; A61K 45/06

(52) U.S. Cl. CPC C12Q 1/6886; A61K 31/4745; A61K 31/52; A61K 31/5377; A61K 31/7056; A61K 31/7076; A61K 45/06; C12Q 2600/106; C12Q 2600/118; C12Q 2600/156; C12Q 2600/158

(57) ABSTRACT

The present disclosure is directed to methods of predicting resistance to a purine analog by detecting the presence of a NT5ex4a isoform. Further provided herein are methods of treating the subject with resistance by administering a purine biosynthesis inhibitor.

This application claims the benefit of U.S. Provisional Patent Application No. 63/351,244 filed Jun. 10, 2022, which is incorporated herein by reference in its entirety.

### SEQUENCE LISTING INCORPORATION

This application contains a Sequence Listing XML, which has been submitted electronically and is hereby incorporated by reference in its entirety. Said XML Sequence Listing, created on Jun. 6, 2023, is named CHOPP0059WO.xml and is 21,986 bytes in size.

### BACKGROUND

### 1. Field of the Disclosure

The present disclosure relates generally to the fields of molecular biology and immunotherapy. More particularly, the disclosure relates to methods for detecting patients with resistance to therapy and methods of treatment thereof.

### 2. Background

B-lymphoblastic leukemia (B-ALL) is a heterogeneous, chromosome translocation-driven disease where the prevalence of somatic mutations and copy number variations is relatively low. Previous B-ALL whole exome sequencing efforts by other groups have focused upon mutations acquired under therapeutic pressure, but they have not identified universal resistance gene(s). Instead, relapse-specific mutations occurred in multiple genetic loci often involved in resistance to either glucocorticoids or purine analogs (e.g., 6-mercaptopurine, or 6-MP). The most prevalent target, NT5C2, encodes the enzyme 5′-nucleotidase/cytosolic II involved in 6-MP catabolism, but these gain-of-function mutations were found in a minority of relapsed/refractory (r/r) B-ALL samples (˜25%). This lack of concordance between the genotype and the phenotype suggested a possibility that instead of mutations, NT5C2 is predominantly affected by post-transcriptional events, such as aberrant mRNA splicing (AS). However, there is an unmet need to identify post-transcriptional modifications associated with resistance.

### SUMMARY

In a first embodiment, there is provided a method of predicting resistance of a cancer patient to a purine analog comprising assaying a cancer cell isolated from the patient to determine the presence of alternatively spliced isoform NT5ex4a of the NT5C2 gene.

In some aspects, the purine analog is mercaptopurine (6-MP), azathioprine, thioguanine, or fludarabine. In specific aspects, the purine analog is mercaptopurine (6-MP). In some aspects, the purine analog is cladribine, clofarabine, or nelarabine.

In some aspects, if the NT5ex4a isoform is present, then the cancer is predicted to be resistant to the purine analog. In certain aspects, the method further comprises determining the expression of the NT5ex4a isoform and/or the wild-type NT5C2 gene as compared to a control. In some aspects, an increased expression of the NT5ex4a isoform as compared to the wild-type NT5C2 gene (e.g., at least 1.1 fold, 1.5 fold, 2 fold, 3 fold, 4 fold, or 5 fold increased expression of the splicing isoform as compared to the wild-type gene) predicts resistance to the purine analog. In some aspects, the method further comprises identifying the patent as having a cancer that is resistant to the purine analog if the NT5ex4a isoform is present. In certain aspects, the method further comprises identifying the patent as having a cancer that is resistant to the purine analog is the NT5ex4a isoform is present at an increased level as compared to the wild-type NT5C2 gene.

In certain aspects, identifying comprises reporting whether the patient has a cancer that is resistant to the purine analog. In some aspects, reporting comprises preparing a written or an oral report. In further aspects, the method further comprises reporting to the patient, a doctor, a hospital, or an insurance provider. In particular aspects, identifying the patient as having a cancer that is resistant to the purine analog further comprises identifying the patient having the cancer as a candidate for treatment with a purine biosynthesis inhibitor.

In additional aspects, the method further comprises treating the patient with a purine biosynthesis inhibitor. In some aspects, the purine biosynthesis inhibitor is mizoribine (4-carbamoyl-1-β-d-ribofuranosyl imirdozolium).

In some aspects, the method further comprises treating the patient with a kinase inhibitor. In certain aspects, the kinase inhibitor is an inhibitor of ATR, ATM, NM1, DNAPK, SMG1, HUNK, CK1A1, QK, PAK4, or PAK5. In certain aspects, the kinase inhibitor is an inhibitor of ATM. In some aspects, the inhibitor of ATM is AZD1390 or Elimusertib. In some aspects, the inhibitor of ATR is M6620, AZD6738, or BAY1895344.

In some aspects, the cancer is leukemia. In particular aspects, the leukemia is B-lymphoblastic leukemia (B-ALL), Acute lymphoblastic leukemia (ALL), or chronic mycloid leukemia (CML). In some aspects, the cancer is small lymphocyte B lymphoma, chronic lymphocytic leukemia, T lymphoblastic leukemia, or acute myelogenous leukemia.

In certain aspects, the method further comprises administering a second anticancer therapy. For example, the second anticancer therapy is a surgical therapy, chemotherapy, radiation therapy, cryotherapy, hormonal therapy, toxin therapy, immunotherapy, or cytokine therapy.

In some aspects, the cancer cell is from a patient sample. In certain aspects, the patient sample is blood, saliva, urine, or tissue biopsy. In particular aspects, the patient sample is blood.

In particular aspects, the presence of the NT5ex4a isoform is detected by performing RT-PCR, such as using primer sequences SEQ ID NOs:1-4. In certain aspects, the presence of the NT5ex4a isoform is detected by performing Western blot, ELISA, immunoprecipitation, radioimmunoassay, or immunohistochemical assay. In some aspects, the presence of the NT5ex4a isoform is detected by performing mass spectrometry or by sequencing a nucleic acid. In some aspects, determining the expression level comprises performing reverse transcription-quantitative real-time PCR (RT-qPCR), microarray analysis, Nanostring® nCounter assay, picodroplet targeting and reverse transcription, or RNA sequencing. In some aspects, the RNA sequencing is long-read nanopore RNA-sequencing. In some aspects, the patient is a human.

A further embodiment provides a method of predicting relapse of a cancer patient comprising assaying a cancer cell isolated from the patient to determine the presence of alternatively spliced isoform NT5ex4a of the NT5C2 gene.

In some aspects, if the NT5ex4a isoform is present, then the cancer is predicted to relapse. In certain aspects, the method further comprises determining the expression of the NT5ex4a isoform and/or the wild-type NT5C2 gene as compared to a control. In some aspects, an increased expression of the NT5ex4a isoform as compared to the wild-type NT5C2 gene predicts cancer relapse.

In certain aspects, the method further comprises treating the patient with a purine biosynthesis inhibitor, such as mizoribine (4-carbamoyl-1-β-d-ribofuranosyl imirdozolium).

In some aspects, the method further comprises treating the patient with a kinase inhibitor. In certain aspects, the kinase inhibitor is an inhibitor of ATR, ATM, NM1, DNAPK, SMG1, HUNK, CK1A1, QK, PAK4, or PAK5. In certain aspects, the kinase inhibitor is an inhibitor of ATM. In some aspects, the inhibitor of ATM is AZD1390 or Elimusertib. In some aspects, the inhibitor of ATR is M6620, AZD6738, or BAY1895344. In some aspects, the patient is administered a purine biosynthesis inhibitor in combination with a kinase inhibitor.

In particular aspects, the cancer is leukemia. For example, the leukemia is B-lymphoblastic leukemia (B-ALL), Acute lymphoblastic leukemia (ALL), or chronic myeloid leukemia (CML). In some aspects, the cancer is small lymphocyte B lymphoma, chronic lymphocytic leukemia, T lymphoblastic leukemia, or acute myelogenous leukemia.

In additional aspects, the method further comprises administering a second anticancer therapy. In certain aspects, the second anticancer therapy is a surgical therapy, chemotherapy, radiation therapy, cryotherapy, hormonal therapy, toxin therapy, immunotherapy, or cytokine therapy.

In some aspects, the cancer cell is from a patient sample. In particular aspects, the patient sample is blood, saliva, urine, or tissue biopsy. In specific aspects, the patient sample is blood.

In certain aspects, the presence of the NT5ex4a isoform is detected by performing RT-PCR. In some aspects, the presence of the NT5ex4a isoform is detected by performing Western blot, ELISA, immunoprecipitation, radioimmunoassay, or immunohistochemical assay. In certain aspects, the presence of the NT5ex4a isoform is detected by performing mass spectrometry or by sequencing a nucleic acid. In some aspects, determining the expression level comprises performing reverse transcription-quantitative real-time PCR (RT-qPCR), microarray analysis, Nanostring® nCounter assay, picodroplet targeting and reverse transcription, or RNA sequencing. In certain aspects, the RNA sequencing is long-read nanopore RNA-sequencing. In specific aspects, the patient is a human.

Further provided herein is a composition comprising a purine biosynthesis inhibitor (or kinase inhibitor) for use in the treatment of a leukemia or lymphoma in a subject identified to have a NT5ex4a isoform.

In some aspects, the composition is formulated for intratumoral, intravenous, intradermal, intraarterial, intraperitoneal, intralesional, intracranial, intraarticularly, intraprostatic, intrapleural, intratracheal, intraocular, intranasal, intravitreal, intravaginal, intrarectal, intramuscular, subcutaneous, subconjunctival, intravesicular, mucosal, intrapericardial, intraumbilical, or oral administration.

In some aspects, the method further comprises at least a second anticancer therapy. In certain aspects, the second anticancer therapy is chemotherapy, radiation therapy, hormone therapy, immunotherapy or cytokine therapy. In some aspects, the patient has been determined to have a cancer cell comprising increased expression of the NT5ex4a isoform as compared to wild-type NT5C2 gene. In certain aspects, the purine biosynthesis inhibitor is mizoribine (4-carbamoyl-1-β-d-ribofuranosyl imirdozolium).

In some aspects, the method further comprises treating the patient with a kinase inhibitor. In certain aspects, the kinase inhibitor is an inhibitor of ATR, ATM, NM1, DNAPK, SMG1, HUNK, CK1A1, QK, PAK4, or PAK5. In certain aspects, the kinase inhibitor is an inhibitor of ATM. In some aspects, the inhibitor of ATM is AZD1390 or Elimusertib. In some aspects, the inhibitor of ATR is M6620, AZD6738, or BAY1895344. In some aspects, the patient is administered a purine biosynthesis inhibitor in combination with a kinase inhibitor.

Another embodiment provides a method of treating a patient with cancer comprising administering an effective amount of a purine biosynthesis inhibitor (or kinase inhibitor) to said patient, wherein the subject is determined to have a cancer with a NT5ex4a isoform.

In some aspects, the subject is identified to have a cancer with increased expression of the NT5ex4a isoform as compared to the wild-type NT5C2. In certain aspects, the purine biosynthesis inhibitor is mizoribine (4-carbamoyl-1-β-d-ribofuranosyl imirdozolium).

In some aspects, the method comprises treating the patient with a kinase inhibitor. In certain aspects, the kinase inhibitor is an inhibitor of ATR, ATM, NM1, DNAPK, SMG1, HUNK, CK1A1, QK, PAK4, or PAK5. In certain aspects, the kinase inhibitor is an inhibitor of ATM. In some aspects, the inhibitor of ATM is AZD1390 or Elimusertib. In some aspects, the inhibitor of ATR is M6620, AZD6738, or BAY1895344. In some aspects, the patient is administered a purine biosynthesis inhibitor in combination with a kinase inhibitor.

In some aspects, the cancer is leukemia. In certain aspects, the leukemia is B-lymphoblastic leukemia (B-ALL), Acute lymphoblastic leukemia (ALL), or chronic myeloid leukemia (CML), the leukemia or lymphoma is B-lymphoblastic leukemia (B-ALL), Acute lymphoblastic leukemia (ALL), chronic myeloid leukemia (CML), small lymphocyte B lymphoma, chronic lymphocytic leukemia, T lymphoblastic Leukemia, or acute myelogenous leukemia.

In some aspects, the method further comprises administering a second anticancer therapy. For example, the second anticancer therapy is a surgical therapy, chemotherapy, radiation therapy, cryotherapy, hormonal therapy, toxin therapy, immunotherapy, or cytokine therapy.

In some aspects, the presence of the NT5ex4a isoform is detected by performing RT-PCR, such as using SEQ ID NOs:1-4. In some aspects, the presence of the NT5ex4a isoform is detected by performing Western blot, ELISA, immunoprecipitation, radioimmunoassay, or immunohistochemical assay. In certain aspects, the presence of the NT5ex4a isoform is detected by performing mass spectrometry or by sequencing a nucleic acid. In some aspects, the expression level was determined by performing reverse transcription-quantitative real-time PCR (RT-qPCR), microarray analysis, Nanostring® nCounter assay, picodroplet targeting and reverse transcription, or RNA sequencing. In some aspects, the RNA sequencing is long-read nanopore RNA-sequencing. In particular aspects, the patient is a human.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1: Alternative splicing regulates response to cancer treatments.

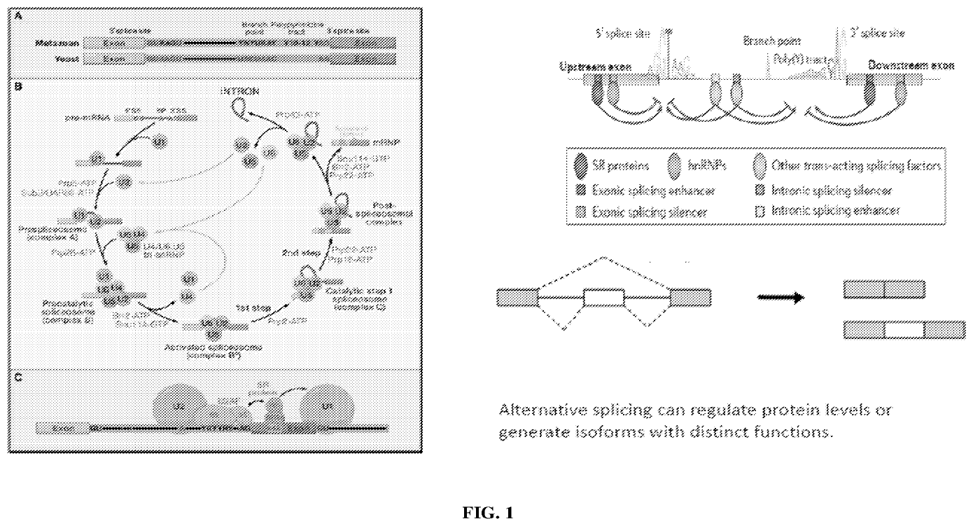

FIG. 2: NT5C2 aberrant splicing in pre- post-relapse paired samples. (Left panel) Heatmap depicting changes in splicing in a cohort of B-ALL paired samples. (Central panel) Boxplot of the PSI values for the diagnostic (pre-) and relapse (post-) samples from the heatmap figure. (Right panel) Gene Ontology (GO) categories deregulated by gene expression on the previous patients during the process of relapse.

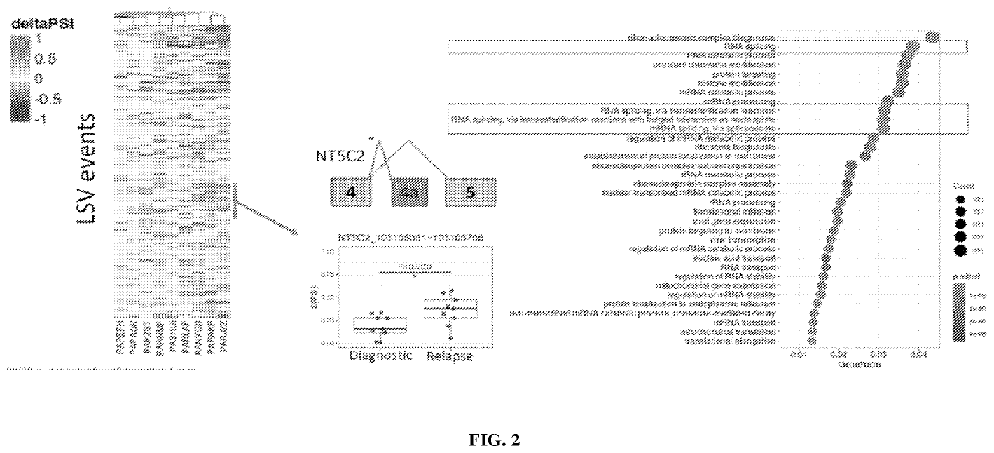

FIGS. 3A-3D: E4a NT5C2 enzymatic activity. (FIG. 3A) Ribbon diagram of the active structure of NT5C2 WT, in which the domain that would harbor E4a region is shown in red. (FIG. 3B) NT5C2 E4a recombinant protein purification workflow. (FIG. 3C) Coomassie staining showing IPTG-induced E4a protein induction in E. coli. NT5C2 band is indicated by an arrow. (FIG. 3D) In vitro nucleotidase assay assessing the enzymatic activity of wild-type and E4a NT5C2 using increasing concentrations of ATP represented as specific activity.

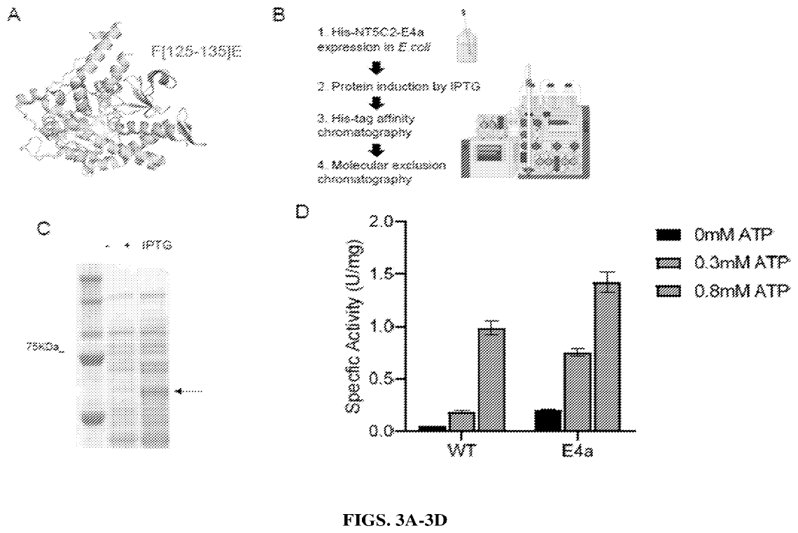

FIG. 4: NT5C2 KO in MHHCALL4. (Top left panel) CRISPR/CAS9 strategy for the generation of total NT5C2 or NT5ex4a isoform specific KOs, representing the exons that will be targeted for each model. (Top right panel) Western-Blot of the NT5C2 levels on the parental, NT5C2-KO and NT5ex4a (E4a) KO models in the 697 and MHHCALL4 cell lines. (Bottom panel) Dose-response plots showing the increased sensitivity to 6-MP treatment upon NT5C2-KO in both cell lines and E4a-KO in the E4a-high expression MHHCALL4 cell line.

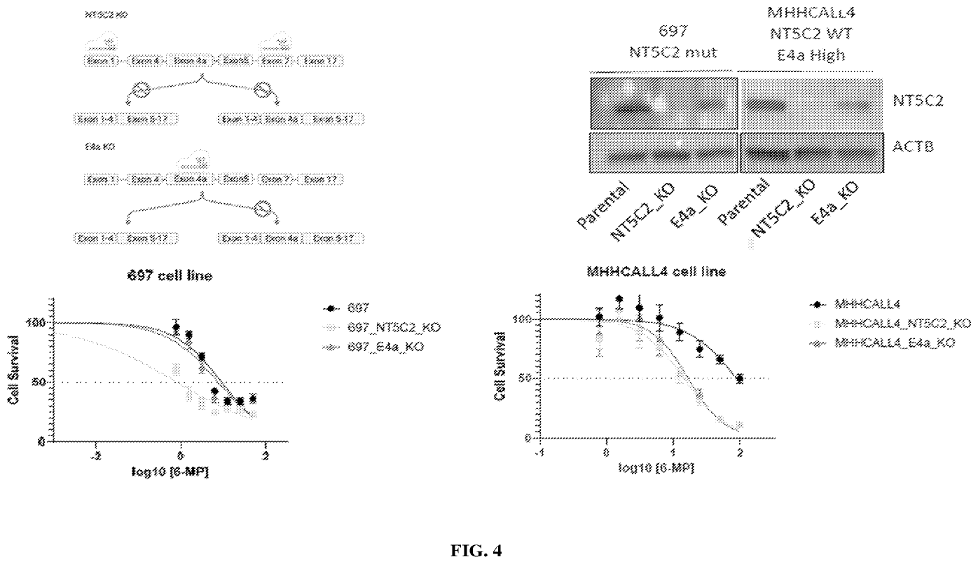

FIG. 5: NT5C2 E4a alternative isoform in 6-MP resistance. (Left panel) Bioluminescent mouse image of mice engrafted with REH cells over-expressing the WT, E4a or R238W NT5C2 isoforms at different time points. (Right panel) Flux quantification of mice engrafted with REH cells over-expressing the WT, E4a or R238W NT5C2 isoforms at different time points.

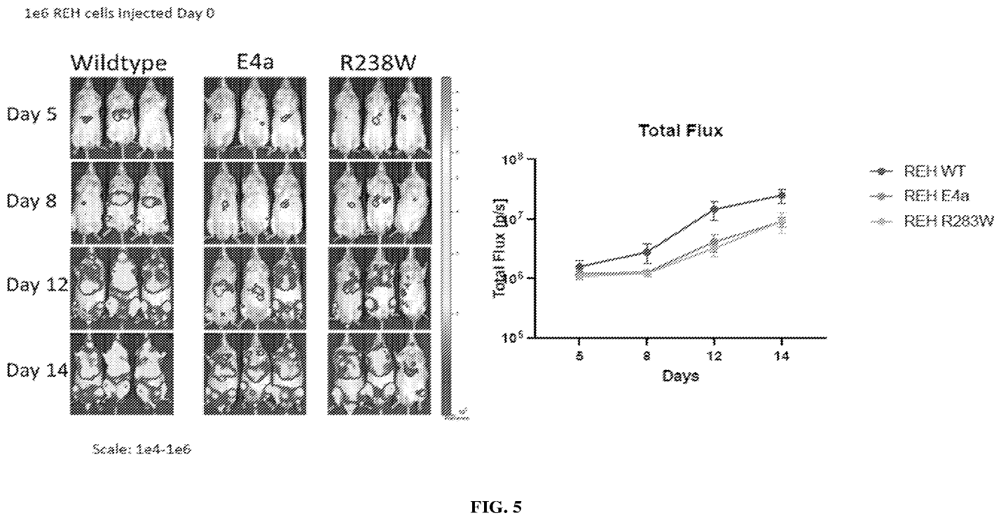

FIG. 6: Mizoribine specifically blocks the IMP and GMP synthases, adding extra pressure in NT5C2 hyperactive cells (Dieck et al., Blood, 2019). (Left panel) Mechanism of action of 6-MP and 6-TG purine analogs. (Right panel) Effects of NT5C2 activating-mutations on purine bases synthesis and degradation.

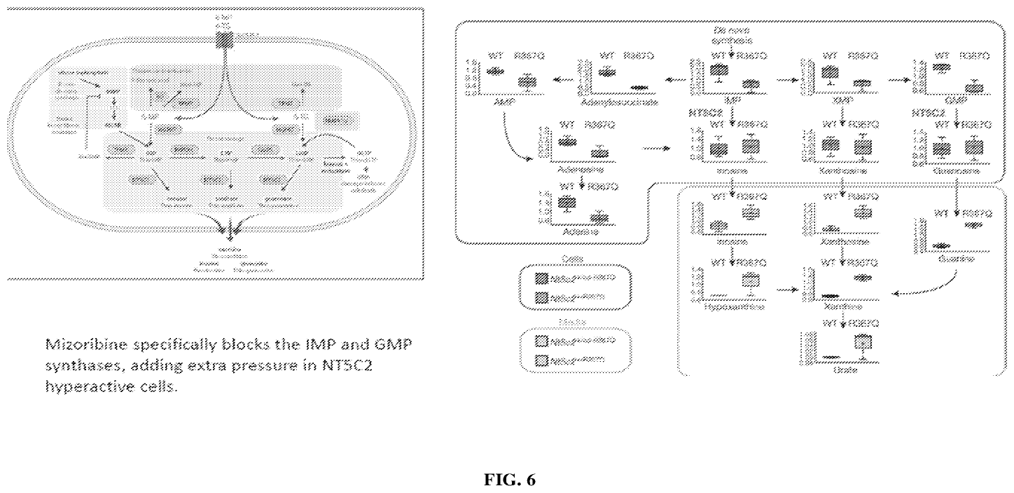

FIG. 7: NT5C2 role in 6-Mercaptopurine (6-MP) or 6-Thioguanine (6-TG) resistance (Dieck et al., Blood, 2019). (Left panel) Mechanism of action of 6-MP and 6-TG purine analogs. (Right panel) NT5C2 activating-mutations found in relapsed leukemia samples.

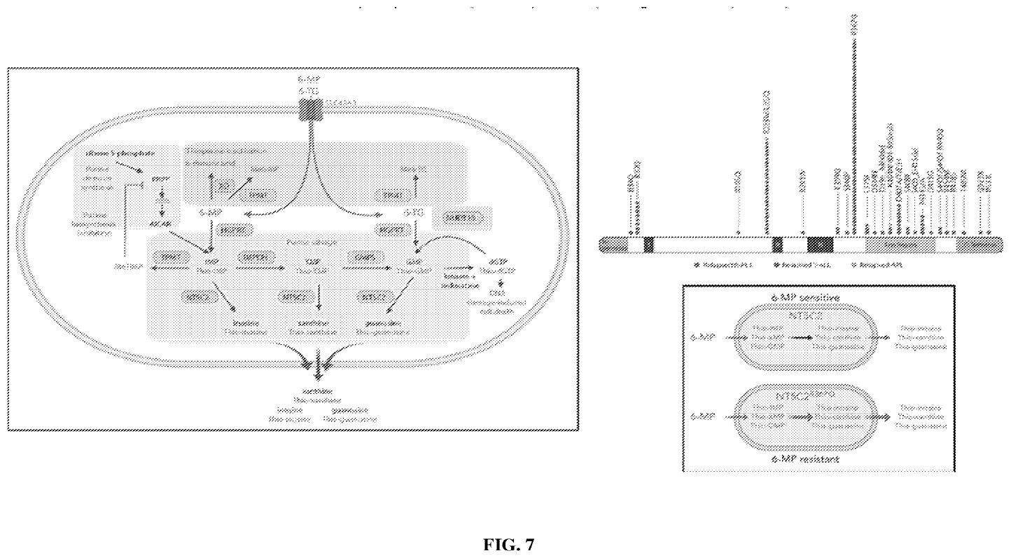

FIGS. 8A-8C: Possible effects of phosphorylation of the NT5C2 activity. (FIG. 8A) Predicted serine and threonine phosphorylation sites in the NT5C2 amino acid sequence. The second row (shaded) shows the site encoded by the cryptic exon NT5ex4a. (SEQ ID NOS: 5-24) (FIG. 8B) Top ten kinases predicted to phosphorylate the NT5C2 exon NT5ex4a-encoded peptide. (FIG. 8C) Increased pro-survival activity of NT5C2 E4a isoform where the Ser-121 has been replaced by a phosphomimetic aspartic acid (S121D). Compare curves with rectangles and triangles.

### DESCRIPTION OF ILLUSTRATIVE EMBODIMENTS

As discussed above, there remains a need for identifying NT5C2 post-transcriptional events which results in therapy resistance, such as to purine analogs.

The present studies analyzed several richly annotated RNA-Seq datasets, including NCI TARGET, which presently includes several hundred baseline childhood B-ALL samples as well as 48 paired diagnostic and relapse samples. In baseline B-ALL samples, an abnormally spliced NT5C2 mRNA isoform was discovered containing the cryptic in-frame exon 4a (NT5ex4a). Of note, NT5ex4a levels were further increased in relapses compared to diagnostic samples, consistent with its putative role in chemoresistance. Furthermore, NT5ex4a mapped to full-length protein-coding transcripts and resulted in inclusion of 8 extra amino acids near the ATP-binding effector site 2.

Using bacterially produced protein, it was demonstrated that at low concentrations of ATP NT5ex4a possessed elevated enzymatic activity compared to the canonical isoform. Consistent with this biochemical finding, in reconstituted NT5C2low B-ALL cells NT5ex4a conferred the same level of resistance to 6-MP as the variant with the R238W hotspot mutation (2-log difference in IC50). Conversely, CRISPR/Cas9 engineered NT5ex4a KO cells exhibited reduced cell survival in the presence of 6-MP. The role of NT5ex4a in chemoresistance in vivo was further confirmed in xenografted B-ALL cells expressing this alternative isoform. Therefore, inclusion of NT5C2 exon 4a phenocopies relapse-specific mutations and could serve as both a valuable predictive biomarker in B-ALL and potentially chronic myelogenous (CML) and acute mycloid leukemia (AML). Additionally, at least in vitro, expression of this non-canonical isoform conferred collateral sensitivity to the purine biosynthesis inhibitor mizoribine, suggesting the existence of a therapeutic window to treat leukemias with dysregulated splicing.

Further, NT5C2 E4a can result in chemoresistance to other FDA-approved purine analogs such as, but not limited to, cladribine, thioguanine, clofarabine, nelarabine, and fludarabine in various cancer models, including but not limited to, small lymphocyte B lymphoma, chronic lymphocytic leukemia, T lymphoblastic Leukemia, and acute myelogenous leukemia. Furthermore, the present methods can be used in non-cancerous pathological conditions such as gastrointestinal disorders including but not limited to ulcerative colitis, Crohn's disease, and inflammatory bowel disease (IBD).

These and other aspects of the disclosure are described in detail below.

### I. ALTERNATIVELY SPLICED ISOFORM DETECTION

In certain embodiments, the method comprises the steps of obtaining a biological sample from a mammal to be tested and detecting the presence of the NT5ex4a isoform in the sample. The NT5C2 gene is alternative spliced resulting in the production of the NT5ex4a isoform.

In one embodiment, the biological sample is a cell sample from a tumor in the mammal. In another embodiment, the biological sample is a circulating tumor cell isolated from the mammal. As used herein the phrase “selectively measuring” refers to methods wherein only a finite number of protein or nucleic acid (e.g., mRNA) markers are measured rather than assaying essentially all proteins or nucleic acids in a sample. For example, in some aspects “selectively measuring” nucleic acid or protein markers can refer to measuring no more than 100, 75, 50, 25, 15, 10, 5, or 2 different nucleic acid or protein markers.

The assays can identify a biomarker for predicting therapy response to a therapeutic regimen. Assays for response prediction may be run before start of therapy and patients showing levels of a biomarker above or below a threshold level of the biomarker are eligible to receive therapy.

### A. Biological Samples

The sample obtained from an individual may contain cells affected by the disease, meaning that the cells express the disease-associated protein. Thus, where the protein is expressed in a cell-specific manner, the sample will contain the cell type in which the disease protein is expressed. The sample analyzed may be any body fluid sample, such as, for example, blood serum, cerebrospinal fluid, mucus, saliva, vaginal secretion, and urine, or may be a sample of the diseased tissue itself.

### 1. Tumor Cell Sample

The method includes collecting samples from a cancer patient for assessment of biomarker levels. The method can use a patient tissue sample of any type or a derivative thereof, including peripheral blood, serum or plasma fraction from peripheral blood, tumor or suspected tumor tissues (including fresh frozen and fixed or paraffin embedded tissue), cell isolates such as circulating epithelial cells separated or identified in a blood sample, lymph node tissue, bone marrow and fine needle aspirates. The sample suitable for use in the method can comprise any tissue type or cell isolates from any tissue type, including a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a serum or plasma fraction of a peripheral blood sample, a tumor tissue or a suspected tumor tissue, a thin layer cytological sample, a fine needle aspirate sample, a bone marrow sample, a lymph node sample, a urine sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample. For example, a patient peripheral blood sample can be initially processed to extract an epithelial cell population, a plasma fraction or a serum fraction, and this extract, plasma fraction or serum fraction can then be assayed. A microdissection of the tissue sample to obtain a cellular sample enriched with suspected tumor cells can also be used. The tissue sample can be processed by any desirable method for performing protein-based assays.

### 2. Circulating Tumor Cells

Circulating tumor cells (CTCs) from any suitable sample type may be used to detect the biomarkers of the present embodiments. The sample may be any sample that includes CTCs suitable for detection of a biomarker. Sources of samples include whole blood, serum, bone marrow, pleural fluid, peritoneal fluid, central spinal fluid, urine, saliva and bronchial washes. In one aspect, the sample is a blood sample, including, for example, whole blood or any fraction or component thereof. A blood sample, suitable for use with the present invention may be extracted from any source known that includes blood cells or components thereof, such as venous, arterial, peripheral, tissue, cord, and the like. For example, a sample may be obtained and processed using well known and routine clinical methods (e.g., procedures for drawing and processing whole blood). In one aspect, an exemplary sample may be peripheral blood drawn from a subject with cancer.

The total number of CTCs in a CTC population is dependent, in part, on the initial sample volume. In various aspects, detection of biomarkers in CTCs from a wide range of initial sample volumes is sufficient to provide clinically significant results. As such, the initial sample volume may be less than about 25 μl, 50 μl, 75 μl, 100 μl, 125 μl, 150 1, 175 μl, 200 μl, 225 μl, 250 μl, 300 μl, 400 μl, 500 μl, 750 μl, 1 ml, 2 ml, 3 ml, 4 ml, 5 ml, 6 ml, 7 ml, 8 ml, 9 ml or greater than about 10 ml. In an exemplary aspect, the initial sample volume is between about 100 and 200 μl. In another exemplary aspect, a sample processed as described herein includes greater than about 1, 2, 5, 7, 10, 15, 20, 30, 40, 50, 100, 200, 300, 400, 500, 600, 700, 800, 900, or even 1000 CTCs. As used herein, biomarker detection analysis includes any method that allows direct or indirect isolation of CTCs and may be in vivo or ex vivo. For example, analysis may include, but not limited to, ex vivo microscopic or cytometric detection and visualization of cells bound to a solid substrate, flow cytometry, fluorescent imaging, and the like. In an exemplary aspect, CTCs are isolated using antibodies directed to CTC-specific cell surface markers.

In another embodiment, the CTCs are captured by techniques commonly used to enrich a sample for CTCs, for example those involving immunospecific interactions, such as immunomagnetic capture. Immunomagnetic capture, also known as immunomagnetic cell separation, typically involves attaching antibodies directed to proteins found on a particular cell type to small paramagnetic beads. When the antibody-coated beads are mixed with a sample, such as blood, they attach to and surround the particular cell. The sample is then placed in a strong magnetic field, causing the beads to pellet to one side. After removing the blood, captured cells are retained with the beads. Many variations of this general method are well known in the art and suitable for use to isolate CTCs.

Isolation of CTCs and characterization of biomarkers therein, using the methods of the invention, is useful in assessing cancer prognosis and in monitoring therapeutic efficacy for early detection of treatment failure that may lead to disease relapse. This is because the presence of CTCs has been associated and/or correlated with tumor progression and spread, poor response to therapy, relapse of disease, and/or decreased survival over a period of time. Thus, enumeration of CTCs and characterization of biomarkers therein provide methods to stratify patients for baseline characteristics that predict initial risk and subsequent risk based upon response to therapy.

Accordingly, in another embodiment, the invention provides a method for diagnosing or prognosing cancer in a subject. CTCs isolated according to the methods disclosed herein may be analyzed to diagnose or prognose cancer in the subject. As such, the methods of the present invention may be used, for example, to evaluate cancer patients and those at risk for cancer. In any of the methods of diagnosis or prognosis described herein, either the presence or the absence of one or more indicators of cancer, such as the NT5ex4a isoform, may be used to generate a diagnosis or prognosis.

In one aspect, a blood sample is drawn from the patient and CTCs are analyzed as described herein. Using the method of the invention, the number of CTCs in the blood sample may be determined and the CTCs subsequently analyzed. For example, the cells may be labeled with one or more antibodies that bind to a CTC-specific cell surface marker, such as, for example, cytokeratin or EpCAM, and the antibodies may have a covalently bound fluorescent label. Analysis may then be performed to characterize the CTCs in the sample, and from this measurement.

### B. Detection Methods

In one embodiment, the methods described herein provide for detecting the presence a posttranscriptional modification of a gene (e.g., NT5ex4a isoform of the NT5CT2 gene (RefSeqGene No. NG_042272.1)) in a biological sample obtained from an individual.

In some embodiment of the methods described herein, detecting the presence of a biomarker in a biological sample obtained from an individual comprises determining the presence of a modified polypeptide in the sample. A polypeptide can be detected by any of a number of means known to those of skill in the art, including analytical biochemical methods, such as electrophoresis, capillary electrophoresis, high performance liquid chromatography (“HPLC”), thin layer chromatography (“TLC”), hyperdiffusion chromatography, and the like, or various immunological methods, such as fluid or gel precipitation reactions, immunodiffusion (single or double), immunoelectrophoresis, radioimmunoassay (“RIA”), enzyme-linked immunosorbent assay (“ELISA”), immunofluorescent assays, flow cytometry, FACS, western blotting, and the like.

Techniques for detecting NT5ex4a isoform in a sample include reverse transcription of mRNA, followed by PCR amplification with primers specific for NT5ex4a isoform mRNA (e.g., RT-PCR or quantitative RT-PCR), and finally sequencing of the amplification product. Alternatively, the mRNA may be sequenced directly without the need for amplification. For example, RT-PCR may be performed using the NT5C2 alternative exon 4a primers and NT5CT2 constitutive exons 5-6-7 primers below.

NT5C2 alternative exon 4a
Forward primer
(SEQ ID NO: 1)
CGATGCCTATGGAAACCGCTTGG
Reverse primer
(SEQ ID NO: 2)
CTCTTCTGAACAGCTACCTGAG
NT5C2 constitutive exons 5-6-7
Forward primer
(SEQ ID NO: 3)
TCAACCTACCAGAGACCTACCTG
Reverse primer
(SEQ ID NO: 4)
TCCTGGAACATACTCCGGTAG

The term “determining an expression level” as used herein means the application of a gene specific reagent such as a probe, primer or antibody and/or a method to a sample, for example a sample of the subject and/or a control sample, for ascertaining or measuring quantitatively, semi-quantitatively or qualitatively the amount of a gene or genes, for example the amount of mRNA. For example, a level of a gene can be determined by a number of methods including for example immunoassays including for example immunohistochemistry, ELISA, Western blot, immunoprecipitation and the like, where a biomarker detection agent such as an antibody for example, a labeled antibody, specifically binds the biomarker and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR, serial analysis of gene expression (SAGE), Northern Blot, digital molecular barcoding technology, for example Nanostring:nCounter™ Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene® ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and for example for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

### 1. Isolation of RNA

Aspects of the present disclosure concern the isolation of RNA from a patient sample for use in determining the presence and/or expression level of the NT5ex4a isoform. The patient sample may blood, saliva, urine, or a tissue biopsy. The tissue biopsy may be a tumor biopsy that has been flash-frozen (e.g., in liquid nitrogen), formalin-fixed and paraffin-embedded (FFPE), and/or preserved by a RNA stabilization agent (e.g., RNAlater). In some aspects, isolation is not necessary, and the assay directly utilizes RNA from within a homogenate of the tissue sample. In certain aspects the homogenate of FFPE tumor sample is enzymatically digested.

RNA may be isolated using techniques well known to those of skill in the art. Methods generally involve lysing the cells with a chaotropic (e.g., guanidinium isothiocyanate) and/or detergent (e.g., N-lauroyl sarcosine) prior to implementing processes for isolating particular populations of RNA. Chromatography is a process often used to separate or isolate nucleic acids from protein or from other nucleic acids. Such methods can involve electrophoresis with a gel matrix, filter columns, coated magnetic beads, alcohol precipitation, and/or other chromatography.

### 2. Expression Assessment

In certain aspects, methods of the present disclosure concern measuring expression of the NT5ex4a isoform and the wild-type NT5C2 gene as well as one or more reference genes in a sample from a subject with breast cancer. The expression information may be obtained by testing cancer samples by a lab, a technician, a device, or a clinician.

Expression levels of the genes can be detected using any suitable means known in the art. For example, detection of gene expression can be accomplished by detecting nucleic acid molecules (such as RNA) using nucleic acid amplification methods (such as RT-PCR, droplet-based RT amplification, exon capture of RNA sequence library, next generation RNA sequencing), array analysis (such as microarray analysis), or hybridization methods (such as ribonuclease protection assay, bead-based assays, or Nanostring®. Detection of gene expression can also be accomplished using assays that detect the proteins encoded by the genes, including immunoassays (such as ELISA, Western blot, RIA assay, or protein arrays).

The pattern or signature of expression in each cancer sample may then be used to generate a cancer prognosis or classification, such as predicting cancer survival or recurrence. The expression of NT5ex4a isoform and the wild-type NT5C2 gene could be assessed to predict or report prognosis or prescribe treatment options for cancer patients, especially leukemia patients.

The expression of NT5ex4a isoform and the wild-type NT5C2 gene may be measured by a variety of techniques that are well known in the art. Quantifying the levels of the messenger RNA (mRNA) of a gene may be used to measure the expression of the gene. Alternatively, quantifying the levels of the protein product of NT5ex4a isoform and the wild-type NT5C2 gene may be to measure the expression of the genes. Additional information regarding the methods discussed below may be found in Ausubel et al., (2003) Current Protocols in Molecular Biology, John Wiley &amp; Sons, New York, NY, or Sambrook et al. (1989) Molecular Cloning: A Laboratory Manual, Cold Spring Harbor Press, Cold Spring Harbor, NY. One skilled in the art will know which parameters may be manipulated to optimize detection of the mRNA or protein of interest.

A nucleic acid microarray may be used to quantify the differential expression of the NT5ex4a isoform and the wild-type NT5C2 gene. Microarray analysis may be performed using commercially available equipment, following manufacturer's protocols, such as by using the Affymetrix GeneChip® technology (Santa Clara, CA) or the Microarray System from lncyte (Fremont, CA). Typically, single-stranded nucleic acids (e.g., cDNAs or oligonucleotides) are plated, or arrayed, on a microchip substrate. The arrayed sequences are then hybridized with specific nucleic acid probes from the cells of interest. Fluorescently labeled cDNA probes may be generated through incorporation of fluorescently labeled deoxynucleotides by reverse transcription of RNA extracted from the cells of interest. Alternatively, the RNA may be amplified by in vitro transcription and labeled with a marker, such as biotin. The labeled probes are then hybridized to the immobilized nucleic acids on the microchip under highly stringent conditions. After stringent washing to remove the non-specifically bound probes, the chip is scanned by confocal laser microscopy or by another detection method, such as a CCD camera. The raw fluorescence intensity data in the hybridization files are generally preprocessed with a robust statistical normalization algorithm to generate expression values.

Quantitative real-time PCR (qRT-PCR) may also be used to measure the differential expression of the NT5ex4a isoform and the wild-type NT5C2 gene. In qRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified.

For example, extracted RNA can be reverse-transcribed using a GeneAmp® RNA PCR kit (Perkin Elmer, Calif., USA), following the manufacturer's instructions. In some embodiments, gene expression levels can be determined using a gene expression analysis technology that measures mRNA in solution. Methods of detecting gene expression are described, for example, in U.S. Patent Application Nos. US20140357660, and US20130259858; incorporated herein by reference. Examples of such gene expression analysis technologies include, but not limited to RNAscope™, RT-PCR, Nanostring®, QuantiGene®, gNPA®, HTG®, microarray, and sequencing. For example, methods of Nanostring use labeled reporter molecules, referred to as labeled “nanoreporters,” that are capable of binding individual target molecules. Through the nanoreporters' label codes, the binding of the nanoreporters to target molecules results in the identification of the target molecules. Methods of Nanostring are described in U.S. Pat. No. 7,473,767 (see also, Geiss et al., 2008). Methods may include the RainDance droplet amplification method such as described in U.S. Pat. No. 8,535,889, incorporated herein by reference. Sequencing may include exon capture, such as Illumina targeted sequencing after the generation of a tagged library for next generation sequencing (e.g., described in International Patent Application No. WO2013131962, incorporated herein by reference).

A non-limiting example of a fluorescent reporter probe is a TaqMan® probe (Applied Biosystems, Foster City, CA). The fluorescent reporter probe fluoresces when the quencher is removed during the PCR extension cycle. Multiplex qRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, qRT-PCR is typically performed using a reference standard. The ideal reference standard is expressed at a constant level among different tissues and is unaffected by the experimental treatment. The system can include a thermocycler, laser, charge-coupled device (CCD) camera, and computer. The system amplifies samples in a 96-well format on a thermocycler. During amplification, laser-induced fluorescent signal is collected in real-time through fiber optics cables for all 96 wells and detected at the CCD. The system includes software for running the instrument and for analyzing the data.

To minimize errors and the effect of sample-to-sample variation, RT-PCR can be performed using an internal standard. The ideal internal standard is expressed at a constant level among different tissues, and is unaffected by an experimental treatment. RNAs commonly used to normalize patterns of gene expression are mRNAs for the housekeeping genes GAPDH, 0-actin, and 18S ribosomal RNA.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Heid et al, 1996). Quantitative PCR is also described in U.S. Pat. No. 5,538,848. Related probes and quantitative amplification procedures are described in U.S. Pat. Nos. 5,716,784 and 5,723,591. Instruments for carrying out quantitative PCR in microtiter plates are available from PE Applied Biosystems (Foster City, CA).

The steps of a representative protocol for quantitating gene expression level using fixed, paraffin-embedded tissues as the RNA source, including mRNA isolation, purification, primer extension and amplification are given in various published journal articles (see Godfrey et al., 2000; Specht et al., 2001). Briefly, a representative process starts with cutting about 10μτη thick sections of paraffin-embedded neoplasm tissue samples or adjacent non-cancerous tissue. The RNA is then extracted, and protein and DNA are removed. Alternatively, RNA is isolated directly from a neoplasm sample or other tissue sample. After analysis of the RNA concentration, RNA repair and/or amplification steps can be included, if necessary, and RNA is reverse transcribed using gene specific primers, followed by preparation of a tagged RNA sequencing library, and paired-end sequencing. In another example, the RNA is not reverse transcribed, but is directly hybridized to a specific template and then labeled with oligonucleotides and/or chemical or fluorescent color to be detected and counted by a laser.

Immunohistochemical staining may also be used to measure the differential expression of a plurality of the NT5ex4a isoform and the wild-type NT5C2 gene. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of a plurality of ER- and PR-related genes. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of the NT5ex4a isoform and the wild-type NT5C2 gene. For this, a plurality of antibodies is arrayed and covalently attached to the surface of the microarray or biochip. A protein extract containing the biomarker proteins of interest is generally labeled with a fluorescent dye.

The labeled NT5ex4a isoform and the wild-type NT5C2 gene proteins may be incubated with the antibody microarray. After washes to remove unbound proteins, the microarray is scanned. The raw fluorescent intensity data may be converted into expression values using means known in the art.

Luminex multiplexing microspheres may also be used to measure the differential expression of a plurality of biomarkers. These microscopic polystyrene beads are internally color-coded with fluorescent dyes, such that each bead has a unique spectral signature (of which there are up to 100). Beads with the same signature are tagged with a specific oligonucleotide or specific antibody that will bind the target of interest (i.e., biomarker mRNA or protein, respectively). The target, in turn, is also tagged with a fluorescent reporter. Hence, there are two sources of color, one from the bead and the other from the reporter molecule on the target. The beads are then incubated with the sample containing the targets, of which up to 100 may be detected in one well. The small size/surface area of the beads and the three-dimensional exposure of the beads to the targets allows for nearly solution-phase kinetics during the binding reaction. The captured targets are detected by high-tech fluidics based upon flow cytometry in which lasers excite the internal dyes that identify each bead and also any reporter dye captured during the assay. The data from the acquisition files may be converted into expression values using means known in the art.

In situ hybridization may also be used to measure the differential expression of a plurality of biomarkers. This method permits the localization of mRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an mRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple mRNAs may be detected simultaneously, provided each antisense probe has a distinguishable label. The hybridized tissue array is generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for each biomarker.

### 2. Immunological Methods

The presence of a modified polypeptide can be determined by contacting the sample with an antibody that specifically binds to the modified polypeptide product (e.g., NT5ex4a isoform) and detecting or measuring the formation of the complex between the antibody and the modified polypeptide. An antibody can be labeled (e.g., radioactive, fluorescently, biotinylated or HRP-conjugated) to facilitate detection of the complex. Appropriate assay systems for detecting polypeptides include, but are not limited to, flow cytometry, enzyme-linked immunosorbent assay (ELISA), competition ELISA assays, radioimmuno-assays (RIA), immunofluorescence, gel electrophoresis, western blot, chemiluminescent assays, bioluminescent assays, and immunohistochemical assays using antibodies having specificity for the modified polypeptide. Numerous methods and devices are well known to the skilled artisan for the detection and analysis of the present disclosure. With regard to polypeptides or proteins in test samples, immunoassay devices and methods are often used. These devices and methods can utilize labeled molecules in various sandwich, competitive, or non-competitive assay formats, to generate a signal that is related to the presence or amount of a biomarker of interest. Additionally, certain methods and devices, such as but not limited to, biosensors and optical immunoassays, may be employed to determine the presence or amount of biomarkers without the need for a labeled molecule.

In general, immunological methods include obtaining a sample suspected of containing an antigen, contacting the sample with a first monoclonal antibody that binds the antigen, and contacting the sample with a composition capable of selectively binding or detecting the complex, e.g., a labeled second antibody, under conditions effective to allow immune complex (antigen/antibody) formation. Examples of compositions capable of selectively binding or detecting the antigen include, but are not limited to, antibodies, aptamers, or other ligands that can be labeled using a variety of markers, e.g., biotin/avidin ligand binding arrangement, as is known in the art. One skilled in the art may also use a labeled third antibody.

“Under conditions effective to allow immune complex (antigen/antibody) formation” means that the conditions preferably include diluting the antigens and/or antibodies with solutions such as BSA, bovine gamma globulin (BGG), or phosphate buffered saline (PBS)/Tween. These agents tend to assist in the reduction of nonspecific background. The “suitable” conditions also means that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Contacting the patient sample with the first antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present, i.e., NT5ex4a isoform. After this time, the sample-antibody composition, such as an ELISA plate, dot blot, or western blot, will generally be washed to remove any non-specifically bound antibody species, allowing only those antibodies specifically bound with the antigen to be detected.

The antigen, antibody, or antigen:antibody complex employed in the detection may itself be linked to a detectable label, wherein one would then simply detect this label, thereby allowing the amount of the antigen in the sample to be determined. Alternatively, the first antibody that becomes bound within the antigen may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected. Further methods include the detection of a primary immune complex by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

Immunohistochemical staining may be used to detect the presence of a biomarker. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the expression of a plurality of biomarkers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

The western blot is a method of assaying for the presence of a particular protein within a biological sample. The general methodology of the western blot is comprised of applying the sample to a polyacrylamide gel and separating the proteins through the technique of gel electrophoresis. The proteins, which have been separated into discrete bands, are subsequently transferred to a sheet (e.g., nitrocellulose or PVDF) by way of a blotting chamber. Once the protein bands have been transferred, the blot is treated with antibody specific to the particular antigen of interest; if the antigen is present, the antibody will bind to the antigen. Free antibody is washed away, the blot is treated with a second antibody which is capable of binding to a site on the first antibody, and the blot is rinsed again to remove excess antibody. In order to detect binding, the second antibody may carry a radiolabel or fluorescent label or may be linked to an enzyme as in the ELISA technique. The enzyme linked to the antibody may then in turn react with a substrate applied to the blot which, for example, generates a colored product. In the case of a radiolabel, the bands may be visualized through the technique of autoradiography, where the radioactive blot is exposed to photographic film for a time sufficient to visualize the protein band or bands of interest. In the case of a fluorescent label, the bands may be visualized using an Odyssey imaging system (LI-COR). The presence of very small quantities of antigen may be detected due to the highly sensitive nature of the western blotting technique.

An antibody microarray may also be used to measure the differential expression of a plurality of biomarkers. For this, a plurality of antibodies is arrayed and covalently attached to the surface of the microarray or biochip. A protein extract containing the biomarker proteins of interest is generally labeled with a fluorescent dye. The labeled biomarker proteins are incubated with the antibody microarray. After washes to remove unbound proteins, the microarray is scanned. The raw fluorescent intensity data may be converted into expression values using means known in the art.

As detailed above, immunoassays, in their most simple and/or direct sense, are binding assays. Certain preferred immunoassays are the various types of enzyme-linked immunosorbent assays (ELISAs) and/or radioimmunoassays (RIA) known in the art. Immunohistochemical detection using tissue sections is also particularly useful. However, it will be readily appreciated that detection is not limited to such techniques, and western blotting, dot blotting, FACS analyses, and/or the like may also be used.

### 3. Analytical Biochemical Methods

Alternatively, the presence of NT5ex4a isoform may be detected using analytical biochemical methods, such as, for example, techniques including gel electrophoresis, isoelectric focusing (IEF), cation exchange HPLC, or mass spectrometry. Mass spectrometric analysis has been used for the detection of proteins in serum samples. Mass spectroscopy methods include Surface Enhanced Laser Desorption Ionization (SELDI) mass spectrometry (MS), SELDI time-of-flight mass spectrometry (TOF-MS), Maldi Qq TOF, MS/MS, TOF-TOF, ESI-Q-TOF and ION-TRAP.

In one embodiment of the method, mass spectrometry methods are used to identify proteolytic disease peptides derived from mutant or modified disease proteins. Such methods are efficient, accurate methods to isolate and identify biomolecules and are well suited to separation and identification of proteolytic disease peptides having a disease mutation that may differ by a single amino acid or that may differ with respect to post-translational modifications as compared to a proteolytic non-disease peptide which may be contained in the same sample of a heterozygous individual.

Thus, techniques such as MALDI-TOF mass spectrometry, LC/MS/MS (MRM) or high resolution LC/MS may be used. Preparation of the sample fraction containing the proteolytic peptides may be done in accordance with routine methods. For example, the fraction of the sample containing the proteolytic peptides may be mixed with a UV-absorbing matrix prior to laser irradiation in a mass spectrometer or injected for LC/MS.

Techniques such as tandem mass spectroscopy (MS/MS) and liquid chromatography/mass spectroscopy (LC/MS and LC/MS/MS) can be used to obtain the sequence of individual peptides in the sample. Briefly, in LC/MS, different peptides are separated by a reverse phase column according to their hydrophobicity and sprayed into a mass spectrometer for mass measurement. In LC/MS/MS, the peptide is further fragmented in the mass spectrometer to generate fragments, and one, a few, or all of the fragments can be measured in the mass spectrometer to increase specificity.

Thus, for example, the use of selected reaction monitoring (SRM) mode and multiple reaction monitoring mode (MRM) can be performed using tandem MS methods in order to identify the sequences of the particular peptides contained within the sample. As indicated above, mass spectroscopy methods can also be used to identify differences in post-translational modification between proteolytic peptides containing a disease mutation and non-disease peptides.

If desired, the results obtained with the sample from the individual to be diagnosed may be compared with samples containing known non-disease proteolytic peptides without any mutation/modification and/or with samples containing known disease proteolytic peptides containing known disease mutations/modifications.

### VI. METHOD OF TREATMENT AND METHODS OF DETECTION

In certain embodiments, the present invention provides methods of selecting a treatment for a disease patient as well as methods of treating a patient with a disease with the selected treatment. The disease may be a cancer, such as leukemia. In some aspects, the subject is identified (e.g., having the presence or increased expression of the exon 4a isoform NT5ex4a as compared to the wild-type NT5C2 gene) to be resistant to a purine analog. Purine analogue interferes with purine nucleoside synthesis and metabolism, thereby interfering with the synthesis and function of DNA and RNA. Purine analogues are antimetabolites that mimic the structure of metabolic purines. The subject that is resistant to the purine analog may be sensitive to a purine biosynthesis inhibitor.

As used herein, the term “sample” refers to any sample suitable for the detection methods provided by the present invention. The sample may be any sample that includes material suitable for detection or isolation. Sources of samples include blood, pleural fluid, peritoneal fluid, urine, saliva, malignant ascites, broncho-alveolar lavage fluid, synovial fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer. In some aspects, the biological sample comprises a plurality of cells. In certain aspects, the biological sample comprises fresh or frozen tissue. In specific aspects, the biological sample comprises formalin fixed, paraffin embedded tissue. In some aspects, the biological sample is a tissue biopsy, fine needle aspirate, nipple aspirate, blood, serum, plasma, cerebral spinal fluid, urine, stool, saliva, circulating tumor cells, exosomes, or aspirates and bodily secretions, such as sweat.

“Prognosis” refers to a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression-free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis and/or cancer progression in a patient susceptible to or diagnosed with a cancer. Prognosis also includes prediction of favorable survival following cancer treatments, such as a conventional cancer therapy.

The term “subject” or “patient” as used herein refers to any individual to which the subject methods are performed. Generally, the patient is human, although as will be appreciated by those in the art, the patient may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

Likewise, an effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The materials identified by the disclosed methods will be useful in treating cancers. Types of cancers to be treated with the binding agents of the disclosure include, but are not limited to, hematological cancers, solid tumors, and non-solid tumors. Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, meningioma, neuroblastoma, retinoblastoma and brain metastases). Adult tumors/cancers and pediatric tumors/cancers are also included.

The subject/patient may be an animal or any species of mammal, including, without limitation, a horse, a dog, a cat, a pig, or a primate. In a particular embodiment, the subject/patient is a human.

In certain embodiments, the compositions identified by the presently disclosed methods may be used in combination with at least one additional therapy. The additional therapy may be radiation therapy, surgery (e.g., lumpectomy and a mastectomy), chemotherapy, gene therapy, DNA therapy, viral therapy, RNA therapy, another immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An additional therapy may be administered before, during, after, or in various combinations relative to the T Cell Receptor Therapy described herein. The administrations may be in intervals ranging from concurrently to minutes to days to weeks. In some embodiments where the therapies are provided to a patient separately, one would generally ensure that a significant period of time did not expire between the time of each delivery, such that the two treatments would still be able to exert an advantageously combined effect on the patient. In such instances, it is contemplated that one may provide a patient with the both therapies within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below a purine biosynthesis inhibitor or kinase inhibitor is “A” and another anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any therapy of the present embodiments to a patient will follow general protocols for the administration of such compounds, taking into account the toxicity, if any, of the agents. Therefore, in some embodiments there is a step of monitoring toxicity that is attributable to combination therapy.

Chemotherapy. A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammaII and calicheamicin omegaII); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazinc, plicomycin, gemcitabien, navelbine, farnesyl-protein tansferase inhibitors, transplatinum, and pharmaceutically acceptable salts, acids, or derivatives of any of the above.

Radiotherapy. Other factors that cause DNA damage and have been used extensively include what are commonly known as 7-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Immunotherapy. The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons ÿ, ÿÿ and ÿ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indolcamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### VII. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Non-Canonical Exon Usage in Pediatric Leukemia

The present studies analyzed several richly annotated RNA-Seq datasets, including NCI TARGET, which presently includes several hundred baseline childhood B-ALL samples as well as 48 paired diagnostic and relapse samples. In baseline B-ALL samples, an abnormally spliced NT5C2 mRNA isoform was discovered containing the cryptic in-frame exon 4a (NT5ex4a). Of note, NT5ex4a levels were further increased in relapses compared to diagnostic samples, consistent with its putative role in chemoresistance. Furthermore, NT5ex4a mapped to full-length protein-coding transcripts and resulted in inclusion of 8 extra amino acids near the ATP-binding effector site 2.

Using bacterially produced protein, it was demonstrated that at low concentrations of ATP NT5ex4a possessed elevated enzymatic activity compared to the canonical isoform. Consistent with this biochemical finding, in reconstituted NT5C2low B-ALL cells NT5ex4a conferred the same level of resistance to 6-MP as the variant with the R238W hotspot mutation (2-log difference in IC50). Conversely, CRISPR/Cas9 engineered NT5ex4a KO cells exhibited reduced cell survival in the presence of 6-MP. The role of NT5ex4a in chemoresistance in vivo was further confirmed in xenografted B-ALL cells expressing this alternative isoform. Therefore, inclusion of NT5C2 exon 4a phenocopies relapse-specific mutations and could serve as both a valuable predictive biomarker in B-ALL and potentially chronic myelogenous (CML) and acute myeloid leukemia (AML). Additionally, at least in vitro, expression of this non-canonical isoform conferred collateral sensitivity to the purine biosynthesis inhibitor mizoribine.

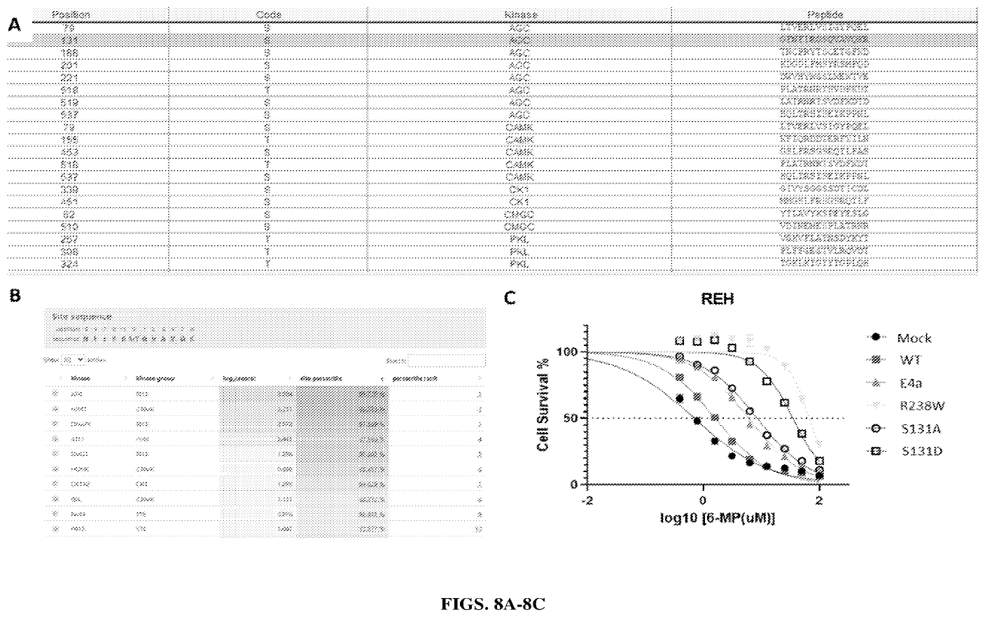

To determine the mechanism underlying the pro-survival activity of the NT5C2 E4a isoform, it was considered that it might be phosphorylated. Indeed, the application of predictive algorithms identified serine at position 121 as the residue in exon NT5ex4a most likely to be phosphorylated (FIG. 8A). Based on the amino acid context, it most strongly resembles consensus sites for kinases of the AGC, CAMK, and PIKK families. The latter includes such important cancer-related kinases as ATR and ATM (FIG. 8B). To determine what effect phosphorylation might have on NT5C2 E4a function, serine at position 121 was replaced with the well-established phosphomimetic aspartic acid (ASP). Indeed, the corresponding isoform conferred stronger survival advantages in the presence of 6-MP that the parental E4a isoform—or its derivative where serine-121 was replaced with the neutral alanine residue (FIG. 8C). Thus, small-molecule inhibitors of ATR, ATM, and other kinases in FIG. 8B could be used to inhibit NT5C2 E4a function and overcome resistance to 6-MP.

Thus, alternative splicing was found to deregulated in relapse samples of B-ALL when compared to diagnostic samples. This deregulation affected important genes such as NT5C2, driving the expression of an alternative isoform that increases the resistance to purine base analog treatments. The alternative isoform of NT5C2 enhanced the sensitivity to purine bases synthesis inhibition, opening a therapeutic window for relapsed patients with increased expression of this variant.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference.
Ausubel et al., (2003) Current Protocols in Molecular Biology, John Wiley &amp; Sons, New York, NY, or Sambrook et al. (1989) Molecular Cloning: A Laboratory Manual, Cold Spring Harbor Press, Cold Spring Harbor, NY.
Bukowski et al., Clinical Cancer Res., 4(10):2337-2347, 1998.
Davidson et al., J. Immunother., 21(5):389-398, 1998.
Dieck et al., Blood, 2019.
Hanibuchi et al., Int. J. Cancer, 78(4):480-485, 1998.
Hellstrand et al., Acta Oncologica, 37(4):347-353, 1998.
Hollander, Front. Immun., 3:3, 2012.
International Patent Publication No. WO2006/12116
International Patent Publication No. WO2013131962
International Patent Publication No. WO2015016718
Pardoll, Nat Rev Cancer, 12(4): 252-64, 2012.
U.S. Pat. No. 4,870,287
U.S. Pat. No. 5,538,848
U.S. Pat. No. 5,716,784
U.S. Pat. No. 5,723,591
U.S. Pat. No. 5,739,169
U.S. Pat. No. 5,760,395
U.S. Pat. No. 5,801,005
U.S. Pat. No. 5,830,880
U.S. Pat. No. 5,846,945
U.S. Pat. No. 7,473,767
U.S. Pat. No. 8,008,449
U.S. Pat. No. 8,354,509
U.S. Pat. No. 8,735,553
U.S. Patent Publication No. 20110008369
U.S. Patent Publication No. 2014022021
U.S. Patent Publication No. 20140294898
U.S. Patent Publication No. US20130259858
U.S. Patent Publication No. US20140357660

1. A method of predicting resistance of a cancer patient to a purine analog comprising assaying a cancer cell isolated from the patient to determine the presence of alternatively spliced isoform NT5ex4a of the NT5C2 gene.

2.-64. (canceled)

65. A method of treating a patient with cancer comprising administering an effective amount of a purine biosynthesis inhibitor to said patient, wherein the subject is determined to have a cancer with a NT5ex4a isoform.

66. The method of claim 65, wherein the subject is identified to have a cancer with increased expression of the NT5ex4a isoform as compared to the wild-type NT5C2.

67. The method of claim 65, wherein the purine biosynthesis inhibitor is mizoribine (4-carbamoyl-1-β-d-ribofuranosyl imirdozolium).

68. The method of claim 65, wherein the cancer is leukemia.

69. The method of claim 68, wherein the leukemia is B-lymphoblastic leukemia (B-ALL), Acute lymphoblastic leukemia (ALL), or chronic myeloid leukemia (CML).

70. The method of claim 65, further comprising administering a second anticancer therapy.

71. The method of claim 70, wherein the second anticancer therapy is a surgical therapy, chemotherapy, radiation therapy, cryotherapy, hormonal therapy, toxin therapy, immunotherapy, or cytokine therapy.

72. The method of claim 71, wherein second anticancer therapy is a kinase inhibitor.

73. The method of claim 72, wherein the kinase inhibitor is an inhibitor of ATR, ATM, NM1, DNAPK, SMG1, HUNK, CK1A1, QK, PAK4, or PAK5.

74. The method of claim 72, wherein the kinase inhibitor is an inhibitor of ATM.

75. The method of claim 74, wherein the inhibitor of ATM is AZD1390 or Elimusertib.

76. The method of claim 65, wherein the presence of the NT5ex4a isoform is detected by performing RT-PCR.

77. The method of claim 65 wherein the presence of the NT5ex4a isoform is detected by performing Western blot, ELISA, immunoprecipitation, radioimmunoassay, or immunohistochemical assay.

78. The method of claim 65, wherein the presence of the NT5ex4a isoform is detected by performing mass spectrometry or by sequencing a nucleic acid.

79. The method of claim 66, wherein the expression level was determined by performing reverse transcription-quantitative real-time PCR (RT-qPCR), microarray analysis, Nanostring® nCounter assay, picodroplet targeting and reverse transcription, or RNA sequencing.

80. method of claim 79, wherein the RNA sequencing is long-read nanopore RNA-sequencing.

81. (canceled)

82. The method of claim 1, wherein the cancer is leukemia or lymphoma.

83. The method of claim 82, wherein the leukemia or lymphoma is B-lymphoblastic leukemia (B-ALL), Acute lymphoblastic leukemia (ALL), chronic myeloid leukemia (CML), small lymphocyte B lymphoma, chronic lymphocytic leukemia, T lymphoblastic Leukemia, or acute myelogenous leukemia.

84. A method of treating a patient with a gastrointestinal disease comprising administering an effective amount of a purine biosynthesis inhibitor to said patient, wherein the subject is determined to have a NT5ex4a isoform.

85.-87. (canceled)
